(19) United States
(12) Patent Application Publication
(10) Pub. No.: US 2004/0229943 A1
(43) Pub. Date: Nov. 18, 2004

(54) Analeptic and drug combinations

(75) Inventors: Rodney J. Hughes, Kennett Square, PA (US); Jeffry L. Vaught, Glenmoore, PA (US)

Correspondence Address: CEPHALON, INC., 145 BRANDYWINE PARKWAY, WEST CHESTER, PA, 19380-4245, US

(73) Assignee: Cephalon Inc, West Chester, PA

(21) Appl. No.: 10/845,836

(22) Filed: May 14, 2004

Related U.S. Application Data
(60) Provisional application No. 60/471,302, filed on May 16, 2003.

(51) Int. Cl. A61K 31/343; A61K 31/165

(52) U.S. Cl. 514/469; 514/617; 514/649; 514/220; 514/259.31; 514/221

(57) ABSTRACT

Compositions and methods for the treatment of disorders through the administration of modafinil with M-drugs.

### BACKGROUND OF THE INVENTION

1. Modafinil

Modafinil, C₁₅H₁₅NO₂S, also known as 2-(benzhydrylsulfinyl) acetamide, or 2-[(diphenylmethyl) sulfinyl] acetamide, is a synthetic acetamide derivative with wake-promoting activity, the structure of which has been described in French Patent No. 78 05 510 and in U.S. Pat. No. 4,177,290 ('290), and which has been approved by the United States Food and Drug Administration for use in the treatment of excessive daytime sleepiness associated with narcolepsy. A method of preparation of a racemic mixture is described in the '290 patent and a method of preparation of a levorotatory isomer is described in U.S. Pat. No. 4,927,855 (both incorporated herein by reference). The levorotatory isomer is reported to be useful for treatment of hypersomnia, depression, Alzheimer's disease and to have activity towards the symptoms of dementia and loss of memory, especially in the elderly.

The primary pharmacological activity of modafinil is to promote wakefulness. Modafinil promotes wakefulness in rats (Touret et al., 1995; Edgar and Seidel, 1997), cats (Lin et al., 1992), canines (Shelton et al., 1995) and non-human primates (Hernant et al, 1991) as well as in models mimicking clinical situations, such as sleep apnea (English bulldog sleep disordered breathing model) (Panckeri et al, 1996) and narcolepsy (narcoleptic canine) (Shelton et al, 1995).

Modafinil has also been described as an agent with activity in the central nervous system, and as a useful agent in the treatment of Parkinson's disease (U.S. Pat. No. 5,180,745); in the protection of cerebral tissue from ischemia (U.S. Pat. No. 5,391,576); in the treatment of urinary and fecal incontinence (U.S. Pat. No. 5,401,776); and in the treatment of sleep apneas and disorders of central origin (U.S. Pat. No. 5,612,379). U.S. Pat. No. 5,618,845 describes modafinil preparations of a defined particle size less than about 200 microns. In addition, modafinil may be used in the treatment of eating disorders, or to promote weight gain or stimulate appetite in humans or animals (U.S. Pat. No. 6,455,588, incorporated herein by reference), or in the treatment of attention deficit hyperactivity disorder (ADHD) (U.S. Pat. No. 6,346,548, incorporated herein by reference), or fatigue, especially fatigue associated with multiple sclerosis (U.S. Pat. No. 6,488,164, incorporated herein by reference).

Modafinil has been shown to be effective in treating narcolepsy, sleepiness, excessive sleepiness (e.g., sleepiness associated with disorders of sleep and wakefulness), excessive daytime sleepiness associated with narcolepsy, Parkinson's disease, urinary incontinence, multiple sclerosis fatigue, ADHD, Alzheimer's disorder, sleep apnea, obstructive sleep apnea, depression, and ischemia.

Narcolepsy is a chronic disorder characterized by intermittent sleep attacks, persistent, excessive daytime sleepiness and abnormal rapid eye movement (“REM”) sleep manifestations, such as sleep-onset REM periods, cataplexy, sleep paralysis and hypnagogic hallucinations, or both (Assoc. of Sleep Disorders Centers, Sleep 2:1 (1979)). Most patients with narcolepsy also have disrupted nocturnal sleep (Montplaisir, in Guilleminault et al. eds., Narcolepsy, Spectrum Pub., New York, pp. 43-56). Pathological somnolence, whether due to narcolepsy or other causes, is disabling and potentially dangerous. Causes of pathological somnolence, other than narcolepsy, include chronic sleep loss (Carskadon et al., Sleep, 5:S73 (1982); Carskadon et al., Psychophysiology, 18:107 (1981)); sleep apnea (Kryger et al., Principles and Practice of Sleep Medicine, W. B. Saunders Co., Philadelphia, Pa. (1989)); and other sleep disorders (International Classification of Sleep Disorders: Diagnostic and Coding Manual, American Sleep Disorder Association, Rochester, Minn. (1990)). Whether due to narcolepsy or other causes, pathological somnolence produces episodes of unintended sleep, reduced attention, and performance errors. Consequently, it is linked to a variety of transportation and industrial accidents (Mitler et al., Sleep 11:100 (1988)). A therapeutic agent that reduces or eliminates pathological somnolence would have important implications not only for individual patients, but also for public health and safety.

Other uses of modafinil have been presented. U.S. Pat. No. 5,180,745 discloses the use of modafinil for providing a neuroprotective effect in humans, and in particular for the therapy of Parkinson's disease. The levorotatory form of modafinil, i.e., (-) benzhydrylsulfinyl-acetamide, may have potential benefit for therapy of depression, hypersomnia and Alzheimer's disease (U.S. Pat. No. 4,927,855). European Published Application 547952 (published Jun. 23, 1993) discloses the use of modafinil as an anti-ischemic agent. European Published Application 594507 (published Apr. 27, 1994) discloses the use of modafinil to treat urinary incontinence.

U.S. Pat. No. RE37,516 discloses pharmaceutical compositions having a defined particle size, and in particular compositions wherein 95% of the cumulative total of the effective amount of modafinil particles in the composition have a diameter less than about 200 microns.

### SUMMARY OF THE INVENTION

In one embodiment, a composition can include therapeutically effective amounts of two or more active compounds, including but not limited to 1) an analeptic, and 2) one or more other drugs. The actives can be combined together and optionally include a pharmaceutically acceptable carrier or the actives can be administered separately.

In another embodiment, the present invention includes using modafinil in combination with one or more other drugs to treat and/or ameliorate one or more symptom associated with a condition which can be treated by the other drug and/or to treat and/or ameliorate one or more side effects associated with treatment or therapy with the other drug.

### DETAILED DESCRIPTION OF THE INVENTION

The present invention is directed to compositions and methods of treating symptoms and side effects associated with various drug therapies and further enhancing the activity of the drug involved in the drug therapy. The symptoms and/or side effects can include but are not limited to narcolepsy, sleepiness, excessive sleepiness (e.g., sleepiness associated with disorders of sleep and wakefulness), excessive daytime sleepiness associated with narcolepsy, urinary incontinence, fatigue, ADHD, sleep apnea, obstructive sleep apnea, depression, and ischemia.

The compositions and methods of the present invention include an analeptic, including but not limited to modafinil, and at least one other drug that has adverse side effects associated with its administration, especially fatigue, sleepiness, sad mood—lack of pleasure, anxiety, worry, irritability, agitation, excessive sleepiness, somnolence, sedation, low energy, lack of motivation, and difficulty in thinking, concentrating and/or remembering.

1. Analeptic Agents

Analeptics are drugs that principally act as or are used as a central nervous system stimulant. Preferred for use in the practice of the invention are analeptics that operate on the sleep-wake centers of the brain and that lack the pharmacological effects of amphetamines. Preferred analeptic agents have the pharmacological profile of modafinil. Thus, in a preferred embodiment of the invention, the analeptic used in the practice of the invention is Provigil® (modafinil).

2. Other Drugs

Any drug that induces or is known to cause as a side effect of its administration, either directly or indirectly, one or more of narcolepsy, sleepiness, excessive sleepiness (e.g., sleepiness associated with disorders of sleep and wakefulness), excessive daytime sleepiness associated with narcolepsy, urinary incontinence, fatigue, ADHD, sleep apnea, obstructive sleep apnea, depression, and/or ischemia, can be used with the present invention.

For example, antipsychotics such as risperidone, clozapine and olanzapine can be used. Further, cholinesterase inhibitors such as donepezil, galantamine, and interferons such as interferon beta-1a and interferon beta-1b can be used. Also, included within the scope of acceptable drugs are dopamine agonists such as ropinirole, bromocriptine, pergolide, and pramipexole. Antiepileptics such as tiagabine, topiramate, phenytoin, carbamazepine, lamotrigine, and gabapentin are also considered within the scope of the present invention. Additionally, heart failure medications such as digoxin, captopril, enalapril, verapamil, diltiazem, nifedipine, losartan, prazosin, and labetalol can also be used. Furthermore, antineoplastics such as taxol, docetaxel, cisplatinum, and vincristine can be used. Generally, the particular drugs and types or classes of drugs suitable for adjunct therapy with modafinil are referred to herein as “modafinil adjunct drugs” or “M-drugs.” An M-drug can include, but is not limited to, a compound set forth above.

3. Variants, Analogs, Salts, Different Forms

Drugs not listed above, including but not limited to structural analogs of the above compounds, that are safe and effective, are also useful in the practice of the invention.

Included within the scope of this invention are the various individual stereoisomers, including diastereomers and enantiomers (e.g., the L and/or R-isomer of modafinil) as well as mixtures thereof. In addition, compounds useful in this invention also include any pharmaceutically acceptable salts, for example: alkali metal salts, such as sodium and potassium; ammonium salts; monoalkylammonium salts; dialkylammonium salts; trialkylammonium salts; tetraalkylammonium salts; and tromethamine salts. Hydrates, solvates, and polymorphs of the compounds described above are included within the scope of this invention. Combinations of analeptics and of M-drugs can also be employed. The compounds can be substantially pure or mixed with other ingredients.

4. Treatment Disorders

The invention is useful in the treatment of disorders and/or side effects associated with an M-drug therapy, including fatigue and sleepiness that may be caused by any of a number of factors, including, for example, depression associated with alcohol or drug abuse. The invention is also useful in the treatment of other disorders for which such M-drugs are sometimes prescribed. These include, for example, epilepsy, heart failure, and psychosis. Such disorders, for which the drugs and types of drugs described herein have been shown to have clinically beneficial effects, are herein referred to collectively as “disorders.”

5. Therapeutically Effective Amounts of Analeptics and M-Drugs

In one embodiment of the present invention, an amount of analeptic, e.g. modafinil, administered to a patient can include from about 5 to 400 mg., more preferably 5, 10, 15, 20, 30, 40, 50, 60, 70, 75, 80, 90, 100, 200, 300 and/or 400 mg. of modafinil, or combinations thereof. Typically, modafinil can be administered in 50, 75, 100 and 200 mg. amounts. However, when used in combination with one or more M-drugs, as described herein, the amount of modafinil necessary to alleviate all or a portion of the symptoms associated with M-drug therapy can be reduced. Accordingly, one embodiment of the present invention includes 100 mg. or less of modafinil when administered with an M-drug, either as a combined unit dose with the M-drug or as a separate dose. A single unit dose containing both modafinil and an M-drug is a preferred composition of the present invention, as described below.

Typically, one or more M-drugs can be administered in the amounts known to be effective for that particular drug or type of drug. More specifically, in the present invention, a drug can be administered in an amount effective to alter the state of an animal subject, i.e., the amount of the M-drug that would be administered to the animal subject if the M-drug was administered alone. Suitable amounts are typically ion the range of 0.1 to 1,000 mg, depending upon the selection of M-drug. For most M-drugs, the amount can be 5, 10, 15, 20, 30, 40, 50, 60, 70, 80, 90, 100, 200, 300 and/or 400 mg. of a particular M-drug, or combinations thereof. However, in the present invention, when used in combination with one or more analeptics such as modafinil, the overall amount of an administered M-drug can be reduced by 10%, 20%, 30%, 40%, 50%, 60%, 70% or 80%, while still providing a therapeutic effect. Accordingly, one embodiment of the present invention includes administering less than an amount of M-drug relative to the amount of M-drug administered to an animal subject if administered alone.

Generally, for daily oral doses of active compounds, the combined total of one or more analeptics and one or more M-drugs will be from about 0.01 mg/kg per day to about 100 mg/kg per day. It is expected that IV doses in the range of about 1 to 1000 mg/cm³ per day will be effective.

In some embodiments of the present invention, the respective weight ratio of analeptic to M-drug can be from 0.01:1 to 1:1 to 100:1, possibly 1000:1. In some embodiments the weight ratio can be 1:1 to 7:1 or 10:1, most preferably 1:1 to 5:1.

A dosage form containing an above described amount of an analeptic (e.g., modafinil) and one or more M-drugs can provide to a patient improved fatigue symptoms, as well as improve waking functioning, as demonstrated by the effects of fatigue, energy, alertness and cognitive function (e.g. psychomotor retardation).

6. Preparation of a Composition of the Present Invention

To prepare a pharmaceutical composition of this invention, an analeptic, including but not limited to modafinil, and an M-drug, including but not limited to one or more of the modafinil adjunct drugs described above, can be intimately admixed. The mixture can further optionally include a pharmaceutical carrier according to conventional pharmaceutical compounding techniques, which carrier may take a wide variety of forms depending on the form of preparation desired for administration, e.g., oral, by suppository, or parenteral. The amount of each active component in the composition can correspond to the amounts described above. Pharmaceutically acceptable carriers include, e.g., stabilizers binders, fillers, disintegrants, lubricants, coatings, sweeteners, flavors, colors, diluents, etc. Such a composition, when used for the therapy of a depressive disorder preferably can include therapeutically effective amounts of an analeptic and M-drug.

In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques.

For parenterals, the carrier will usually comprise sterile water, though other ingredients, for example, for purposes such as aiding solubility or for preservation, may be included. Injectable suspensions may also be prepared in which case appropriate liquid carriers, suspending agents and the like may be employed.

In one embodiment, a pharmaceutical composition of the present invention can be administered in a tablet or capsule form or other suitable unit dose form. A tablet or capsule of the present invention can contain one or more of the following inactive ingredients: lactose hydrous, pregelatinized starch, microcrystalline cellulose, sodium starch glycolate, magnesium stearate, purified water, camauba wax, hydroxypropyl methylcellulose, titanium dioxide, polyethylene glycol, synthetic iron oxide, and polysorbate 80.

Accordingly, a pharmaceutical compositions herein will contain, per dosage unit, e.g., tablet, capsule, powder injection, teaspoonful, suppository and the like from about 5 to about 1000 mg, or more, of an analeptic and M-drug. In on embodiment of the invention, each single dosage unit (or unit dose) includes both an amount of an analeptic and an amount of an M-drug. In such an embodiment, it is not necessary that each single dosage unit include an effective amount so long as the total amount of drug administered to a patient is an effective amount of each. Therefore, for example, a patient may require 2 or more single dosage units to receive effective amounts of both agents.

When administered, the formulations of the invention are applied in pharmaceutically acceptable amounts and in pharmaceutically acceptable compositions. Such preparations may routinely contain salts, buffering agents, preservatives, compatible carriers, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, p-toluene sulfonic, tartaric, citric, methane sulfonic, formic, malonic, succinic, naphthalene-2-sulfonic, and benzene sulfonic. Also, pharmaceutically acceptable salts can be prepared as alkaline metal or alkaline earth salts, such as sodium, potassium or calcium salts.

Suitable buffering agents include: acetic acid and a salt (1-2% W/V); citric acid and a salt (1-3% W/V); boric acid and a salt (0.5-2.5% W/V); and phosphoric acid and a salt (0.8-2% W/V). Suitable preservatives include benzalkonium chloride (0.003-0.03% W/V); chlorobutanol (0.3-0.9% W/V); parabens (0.01-0.25% W/V) and thimerosal (0.004-0.02% W/V).

Dosage may be adjusted appropriately to achieve desired drug levels, locally or systemically. As noted above, generally, daily oral doses of active compounds will be from about 0.01 mg/kg per day to 2000 mg/kg per day. In the event that the response in a subject is insufficient at such doses, even higher doses (or effective higher doses by a different, more localized delivery route) may be employed to the extent that patient tolerance permits. Continuous IV dosing over, for example 24 hours or multiple doses per day is contemplated to achieve appropriate systemic levels of compounds.

A variety of administration routes are available. The particular mode selected will depend of course, upon the particular drug selected, the severity of the disease state(s) being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, intramuscular, or infusion.

The compositions may conveniently be presented in unit dosage form and may be prepared by any of the methods well known in the art of pharmacy. In general, the compositions are prepared by uniformly and intimately bringing the compounds into association with a liquid carrier, a finely divided solid carrier, or both, and then, if necessary, shaping the product.

Compositions suitable for oral administration may be presented as discrete units such as capsules, cachets, tablets, or lozenges, each containing a predetermined amount of the active compound. Other compositions include suspensions in aqueous liquors or non-aqueous liquids such as a syrup, an elixir, or an emulsion.

Other delivery systems can include time-release, delayed release or sustained release delivery systems. Such systems can avoid repeated administrations of the active compounds of the invention, increasing convenience to the subject and the physician. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. In addition, a pump-based hardware delivery system can be used, some of which are adapted for implantation.

Another embodiment of the present invention provides a kit or device which can facilitate the administration of an amount of an analeptic and an M-drug to treat a disorder. Specifically, a kit according to the present invention includes at least one dosage form containing an analeptic, including but not limited to modafinil, and a separate dosage form containing at least one M-drug. One suitable kit of the present invention includes a blister pack having a unit dose of modafinil and a separate unit dose of an M-drug. Most preferably, the unit dose of modafinil includes a 50, 75, 100 or 200 mg. tablet of modafinil and the unit dose of an M-drug includes a 10, 20, 30, 40 or 50 mg. tablet of antidepressant. The kit or device can also include instructions concerning administration of the analeptic and M-drug. Preferably, the instructions provide administration guidance according to one or more of the administration schemes set forth below.

The analeptic and/or M-drug can be in any suitable dosage form, including but not limited to solid dosage forms including tablets, capsules, pills, troches, cachets, and the like, and/or liquid dosage forms such as an oral elixir or an IV fluid. The dosage form of the analeptic can be the same type or a different type than the M-drug.

In yet another embodiment, the present invention includes a transdermal drug delivery system (“TDDS”). A TDDS suitable for use with the invention in patch form typically contains at least: (1) a backing layer and (2) a carrier formulated with an effective amount of an M-drug and optionally modafinil.

Preferred patches include (1) the matrix type patch; (2) the reservoir type patch; (3) the multi-laminate drug-in-adhesive type patch; and (4) the monolithic drug-in-adhesive type patch; and (Ghosh, T. K.; Pfister, W. R.; Yum, S. I. Transdermal and Topical Drug Delivery Systems, Interpharm Press, Inc. p. 249-297, incorporated herein by reference). These patches are generally available commercially.

For practice of the invention, the matrix type and the drug-in-adhesive type patches are especially preferred. The more preferred drug-in-adhesive patch is the monolithic type.

Transdermal drug delivery systems other than standard patches can also be used. These include, for example, osmotic pump systems, ultrasonic systems, ointments, pastes, gels, medicated powders, creams, lotions, aerosols, sprays, foams, medicated adhesives and the like.

7. Method of Treatment/Therapy

A. Administration Schemes and Timing of Treatment of an Analeptic and M-drug

An analeptic and an M-drug can be combined together into a single unit dose, but can also be administered separately as two or more distinct doses.

Thus, in some embodiments of the invention, a treatment of a disorder related to depression can be through the use of separate dosage forms—one or more analeptic doses and one or more M-drug doses. Accordingly, a dose of an analeptic can be administered at a different time relative to the M-drug dose or simultaneously (i.e., analeptic dose administration within less than 1 hour before or after administration of the M-drug). However, if simultaneous, the administration of the analeptic and M-drug can also be through the use of a single unit dose including both an analeptic and M-drug.

In patients that are beginning M-drug therapy, i.e. patients that are substantially free of M-drugs or patients that have been free of M-drug therapy for about 1 week, 2 weeks, more preferably about 4 or more weeks, the dosage form containing the analeptic can be administered before and/or at about the same time as an initial administration of the M-drug. In such an embodiment, one or more administrations of an analeptic can be within 72 hours, preferably within 48 hours, more preferably within 24 hours, most preferably within 1 hour or moments before an initial administration/dosing of an M-drug. After the initial administration of the analeptic and M-drug, subsequent dosings of the analeptic and M-drug can continue at a typical rate, e.g., typically one or two 50, 75, 100 to 200 mg. doses of modafinil per day and 10, 20, 30, 40 to 50 mg. of M-drug per day. Further, after the initial administration of the M-drug, the dosings of the analeptic and M-drug can be in separate dosage forms or in a single unit dose. However, if a dose of an analeptic is to be administered before a subsequent dose of an M-drug, separate dosage forms for each are preferred.

Additionally, in patients that are substantially free of M-drugs, the initial administration of the analeptic can coincide with or be nearly simultaneous with the initial administration of an M-drug. This can be accomplished through the use of separate dosage forms of an analeptic and M-drug which can then be administered together simultaneously (i.e., within 1 hour or less, before or after the M-drug) or through the use of a single unit dose including both an analeptic and an M-drug, as noted above.

Further, an analeptic, including but not limited to modafinil, can also be administered to a patient that has already received at least an initial dose of an M-drug. In one embodiment, the initial administration of an analeptic can be within 72 hours, preferably within 48 hours, more preferably within 24 hours, most preferably within 1 hour or within moments after the initial administration of an M-drug. In this timing scheme, modafinil is administered at about the same time as an M-drug, but subsequent to at least one administration of an M-drug. After the initial dosing of an analeptic, the dosing of the analeptic and M-drug can continue in a typical manner. In one particularly preferred embodiment, initial administration of an analeptic and subsequent administrations of an analeptic can be accomplished through the use of a single unit dose including both an analeptic and an M-drug.

In a further embodiment, initial administration of an analeptic to a patient can occur and/or continue after M-drug therapy has ended. Preferably, this is accomplished by administering an amount of the analeptic to the patient and the administration of which can continue for 1, 2, 5, 10, 20, or 30 days, or more, after M-drug therapy cessation.

In embodiments where the analeptic and M-drug are in separate dosage forms, the administration of the analeptic can preferably occur within moments, or in less than 1 hour, or less than 5 hours, or less than 24 hours or less than 48 hours, or less than 72 hours before or after administration of the M-drug, unless otherwise indicated by a particular method of treatment below.

In one embodiment, an M-drug can be administered before administration of an analeptic.

B. Administration of an Analeptic To Reduce Side Effects Associated With M-drug Treatment Cessation

Administration of a combination of an analeptic such as modafinil and one or more M-drugs can significantly reduce the adverse side effects associated with the discontinuation of M-drug therapy. In such an embodiment, an effective amount of modafinil can be administered simultaneously with an M-drug and/or after M-drug therapy has been discontinued.

In one embodiment, the present invention includes a method of reducing adverse symptoms in an animal subject associated with the cessation of M-drug therapy. The method includes administering an effective amount of one or more analeptics, including but not limited to modafinil, to the animal subject, preferably a human, to reduce the adverse symptoms, wherein the analeptic is administered before and/or during and/or after M-drug therapy cessation, according to one or more of the timing schemes set forth above. The amount of analeptic and duration of analeptic therapy can vary from subject to subject.

However, in one embodiment, the amount of analeptic includes an effective amount, typically from about 100 mg to about 200 mg of modafinil administered once or twice daily during M-drug therapy. In another embodiment, administration of an analeptic can occur within a period of 2 days, preferably less than 10 days, prior to the cessation of therapy of the M-drug with which it is desired to have a reduction of adverse symptoms. The administration of both modafinil and an M-drug can significantly reduce the adverse side effects associated with the discontinuation of M-drug therapy.

In one embodiment, the analeptic can be administered after M-drug treatment cessation. In such an embodiment, the administration of an analeptic can continue for a period of 1, 2, 5, 10, 20, or 30 days or more after the cessation of M-drug therapy.

In such an embodiment, the modafinil can be administered orally, nasally, rectally, intravenously, epidurally, intraperitoneally, subcutaneously, intramuscularly or intrathecally.

C. Disorder Treatment Generally

In one embodiment, the present invention includes a method of treating a depressive disorder by providing a pharmacologically active composition to a subject in need of the composition, preferably a human subject. The pharmacologically active composition can include an amount of an analeptic, preferably a therapeutically effective amount of an analeptic, and one or more M-drugs. The composition can optionally further include a pharmaceutically acceptable carrier, as described above. The pharmacologically active composition can then be administered to an animal subject.

The analeptics and other drugs can be administered to a patient simultaneously or at different times, as described above, and can follow one or more of the administration schemes set forth above.

The pharmacologically active composition and/or combination can be administered via any acceptable route, including but not limited to orally, nasally, rectally, intravenously, epidurally, intraperitoneally, subcutaneously, intramuscularly or intrathecally.

In one embodiment, the administered analeptic is modafinil or a salt thereof. In another embodiment, the M-drug can include one or more of the M-drugs described above, including but not limited to tiagabine.

In one embodiment, one or more analeptics are in a single unit dose form and one or more M-drugs are in a separate unit dose form. Each unit dose form can be administered simultaneously or at different times, according to one or more of the administration schemes described above. In a preferred embodiment, the pharmacologically active composition contains both an analeptic and an M-drug in a single unit dose form.

In a further embodiment, a therapy method for treating a depressive disorder in an animal subject includes topically applying a pharmaceutically acceptable drug formulation having at least one M-drug to the skin of an animal, wherein the pharmaceutically acceptable drug formulation is contained in a patch. An additional effective amount of one or more analeptics, including but not limited to modafinil, can be provided to the animal, either in a matrix included in the patch or via administration of the analeptic through any other acceptable route, including but not limited to orally, nasally, rectally, intravenously, epidurally, intraperitoneally, subcutaneously, intramuscularly or intrathecally.

D. Reduction of Disorder Side Effects

In one embodiment, the present invention includes a method of treating a subject for depression and other disorders for which M-drugs are indicated, whereby side effects of M-drug therapy are reduced. The method includes the steps of administering to the subject an effective amount of an analeptic agent in addition to administering to the patient an effective amount of an M-drug. The therapy can occur according to one or more timing schemes set forth above.

In another embodiment, the present invention includes a method for enhancing activity of an M-drug, whereby side effects are reduced. The method includes the steps of administering to the subject an effective amount of an analeptic agent in addition to administering to the patient an effective amount of an M-drug according to one or more of the administration schemes set forth above.

In yet another embodiment, the present invention includes a method of decreasing onset time of an M-drug, whereby side effects are reduced. The method includes administering to the subject an effective amount of an analeptic agent in addition to administering to the patient an effective amount of an M-drug according to one or more of the administration schemes set forth above.

In a further embodiment, the present invention includes a method for enhancing activity of an M-drug and decreasing onset time of an M-drug, whereby side effects are reduced. The method includes the steps of administering to the subject an effective amount of an analeptic agent in addition to administering to the patient an effective amount of an M-drug according to one or more of the administration schemes set forth above.

E. Enhancing M-drug Activity

In one embodiment, the present invention includes a method of enhancing the activity of an M-drug in an animal subject, preferably a human. The method includes the step of pre-treating the subject with an effective amount of one or more analeptics, including but not limited to modafinil. The amount of analeptic and duration of pretreatment can vary from subject to subject, but typically conforms to the amounts described above and one or more of the timing schemes set forth above.

However, in one particular embodiment, the amount of analeptic includes an effective amount, typically from about 100 mg to about 200 mg of modafinil administered once or twice daily for a period of less than 2 days, preferably less than 10 days, prior to the initiation of M-drug therapy. The administration of the analeptic can also optionally continue during M-drug therapy and also continue for a period of time after the cessation of M-drug therapy, as described above.

The analeptic can be administered orally, nasally, rectally, intravenously, epidurally, intraperitoneally, subcutaneously, intramuscularly or intrathecally.

F. Reduction of Onset Time of M-drug Effect

The time lapse between initiation of M-drug therapy and alleviation of associated M-drug therapy symptoms can be shortened. In one embodiment of the present invention, depressive symptoms can be improved after the initiation of administration of an analeptic, including but not limited to modafinil, before or during M-drug therapy or by following one or more of the timing schemes set forth above.

The time of improvement can be from 1, 2, 4, 7, 10, and 14 days relative to M-drug therapy alone.

In a further embodiment, the present invention includes a method of decreasing the onset time of an M-drug in an animal subject. The method includes the step of pre-treating the subject with an effective amount of one or more analeptics, including but not limited to modafinil and/or co-administering an effective amount of one or more analeptics, including, but not limited to modafinil with an M-drug. The amount of analeptic and duration of pretherapy can vary from subject to subject. However, it is preferred that the timing of administration of the analeptic follow one or more of the timing schemes set forth above.

In one embodiment, the amount of analeptic includes an effective amount of modafinil, typically from about 100 mg to about 200 mg of modafinil administered once or twice daily for a period of less than 2 days, preferably less than 10 days, prior to the initiation therapy of the M-drug with which it is desired to have a decrease in onset time. In another embodiment, the first administration of an analeptic can be within 72 hours, preferably within 48 hours, more preferably within 24 hours, most preferably within 1 hour or within moments before initial administration of an M-drug. As noted above, the administration of the analeptic can also optionally continue during M-drug therapy.

The analeptic can be administered orally, nasally, rectally, intravenously, epidurally, intraperitoneally, subcutaneously, intramuscularly or intrathecally.

G. Reduction of Adverse Symptoms

In one embodiment, the present invention includes a method of reducing adverse symptoms in an animal subject associated with M-drug therapy. The method includes administering an effective amount of one or more analeptics, including but not limited to modafinil, to the animal subject, preferably a human, to reduce the adverse symptoms, wherein the analeptic is administered before and/or during and/or after M-drug therapy or according to one or more administration schemes set forth above.

Adverse symptoms treatable with the therapy of the present invention include, but are not limited to fatigue, sleepiness, sad mood—lack of pleasure, anxiety, worry, irritability, agitation, excessive sleepiness, somnolence, sedation, low energy, lack of motivation, and difficulty in thinking, concentrating and/or remembering. Some or all of these symptoms can be measured using standard Fatigue Severity Scales (FSS), Visual Analogue Scales (VAS) and Epworth Sleepiness Scales (ESS).

The amount of analeptic and duration of analeptic therapy can vary from subject to subject. However, in one embodiment, the amount of analeptic includes from about 100 mg to about 200 mg of modafinil administered once or twice daily 1, 2, 5, 10, 20 or 30 days or more before, during and/or 1, 2, 5, 10, 20, or 30 days or more after cessation of M-drug therapy. Preferably, modafinil administration continues during M-drug therapy.

In certain preferred embodiments, the M-drug includes tiagabine administered at about 20 mg. per day for the duration of M-drug therapy.

In such an embodiment, the modafinil and/or M-drug can be administered orally, nasally, rectally, intravenously, epidurally, intraperitoneally, subcutaneously, intramuscularly or intrathecally.

### Definitions

“Particle,” as used herein, refers to an aggregated physical unit of the acetamide compound, i.e., a piece or a grain of acetamide.

As used herein, “about” means plus or minus ten percent of the indicated value, such that “about 20 mg” indicates 18 to 22 mg.

As used herein, “consisting essentially of” refers to excluding other active ingredients but including excipients and additional amounts of the active ingredient to account for degradation or otherwise.

An “effective amount,” as used herein, is an amount of modafinil and/or M-drug that is effective for treating a depressive state, i.e., an amount of modafinil and/or M-drug that is able to reduce, alleviate or eliminate certain symptoms associated with depression and/or antidepression therapy.

A “pharmaceutical composition,” as used herein, means a medicament for use in treating a mammal that comprises modafinil prepared in a manner that is appropriate for administration to a mammal. A pharmaceutical composition according to the invention may also, but does not of necessity, include a non-toxic pharmaceutically acceptable carrier. A pharmaceutical composition can also include bulk active modafinil for use in preparing dosage forms. A pharmaceutical composition can also include modafinil in combination with another active, preferably and M-drug, more preferably an SSRI.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations. Further, the contents of all references cited herein are hereby incorporated by reference.

What is claimed is:

1. A pharmaceutical composition for the treatment of a depressive disorder comprising an analeptic, an M-drug, and a pharmaceutically acceptable carrier.

2. The composition according to claim 1 wherein the analeptic comprises modafinil.

3. The composition of claim 1 wherein the analeptic is a pharmaceutically acceptable salt of modafinil.

4. The composition of claim 1 wherein the analeptic is a substantially pure enantiomer of modafinil.

5. The composition according to claim 1 wherein the ratio of analeptic to M-drug is from 0.1 to 10:1, by weight.

6. The composition of claim 5, wherein the ratio of analeptic to M-drug is from 1:1 to 5:1, by weight.

7. The composition of claim 1 wherein the M-drug is selected from the group consisting of antipsychotics, cholinesterase inhibitors, interferons, dopamine agonists, antiepileptics, heart failure medications, and antineoplastics.

8. The composition of claim 1 wherein the M-drug is selected from the group consisting of risperidone, clozapine, olanzapine, donepezil, galantamine, interferon beta-1a, interferon beta-1b, ropinirole, bromocriptine, pergolide, pramipexole, tiagabine, topiramate, phenytoin, carbamazepine, lamotrigine, gabapentin, digoxin, captopril, enalapril, verapamil, diltiazem, nifedipine, losartan, prazosin, labetalol, taxol, docetaxel, cisplatinum, and vincristine.

9. The composition of claim 1 wherein the amount of M-drug includes 5, 10, 15, 20, 30, 40, 50, 60, 70, 80, 90, 100, 200, 300 or 400 mg of M-drug.

10. The composition of claim 1 wherein the amount of analeptic includes 5, 10, 15, 20, 30, 40, 50, 60, 70, 75, 80, 90, 100, 200, 300 or 400 mg of analeptic.

11. The composition of claim 4 wherein the enantiomer is the R-isomer of modafinil.

12. The composition of claim 1 wherein the analeptic is administered before or simultaneously with administration of the M-drug.

13. A method for treating a depressive disorder in an animal subject comprising:
(a) providing a pharmacologically active composition comprising therapeutically effective amounts of an analeptic and an M-drug; and
(b) administering the pharmacologically active composition to the subject to treat a depressive disorder wherein the analeptic and M-drug are in a single unit dose or the administration of modafinil is prior to or at about the same time as the administration of the M-drug.

14. The method according to claim 13 wherein the analeptic comprises modafinil.

15. The method of claim 14 wherein the M-drug is selected from the group consisting of antipsychotics, cholinesterase inhibitors, interferons, dopamine agonists, antiepileptics, heart failure medications, and antineoplastics.

16. The method of claim 13 wherein the M-drug is selected from the group consisting of risperidone, clozapine, olanzapine, donepezil, galantamine, interferon beta-1a, interferon beta-1b, ropinirole, bromocriptine, pergolide, pramipexole, tiagabine, topiramate, phenytoin, carbamazepine, lamotrigine, gabapentin, digoxin, captopril, enalapril, verapamil, diltiazem, nifedipine, losartan, prazosin, labetalol, taxol, docetaxel, cisplatinum, and vincristine.

17. The method of claim 13 wherein the amount of M-drug includes 5, 10, 15, 20, 30, 40, 50, 60, 70, 80, 90, 100, 200, 300 or 400 mg of M-drug.

18. The method of claim 13 wherein the amount of analeptic includes 5, 10, 15, 20, 30, 40, 50, 60, 70, 75, 80, 90, 100, 200, 300 or 400 mg of analeptic.

19. A method of reducing adverse symptoms associated with the cessation of M-drug therapy in an animal subject comprising administering an amount of modafinil sufficient to reduce the adverse symptoms to the subject.

20. A method of treating a subject for depression and other disorders for which M-drugs are indicated, whereby side effects are reduced comprising administering an effective amount of an analeptic agent and an M-drug to the subject.

21. A method for decreasing the onset time of an M-drug administered to an animal subject comprising treating the subject with an effective amount of modafinil prior to the administration of the M-drug.

22. The method of claim 21 wherein modafinil is administered one or more of 72 hours, 48 hours, 24 hours, 1 hour or within moments before administration of an M-drug.
